(19) United States
(12) Patent Application Publication
(10) Pub. No.: US 2024/0307472 A1
(43) Pub. Date: Sep. 19, 2024

(54) BEVERAGES CONTAINING NATURAL PRODUCTS

(71) Applicant: PK Beverages LLC, Boca Raton, FL (US)

(72) Inventors: J. Phillip Kennedy, Grand Rapids, MI (US); Karyemaître Aliffe, Olympia, MI (US)

(21) Appl. No.: 18/606,203

(22) Filed: Mar. 15, 2024

Related U.S. Application Data
(60) Provisional application No. 63/490,299, filed on Mar. 15, 2023.

(51) Int. Cl. A61K 36/185; A61K 9/00; A61K 31/00; A61K 31/12; A61K 31/352; A61K 31/375; A61K 31/4525; A61K 31/704; A61K 33/06; A61K 33/08; A61K 33/10; A61K 33/26

(52) U.S. Cl. CPC A61K 36/185; A61K 9/0095; A61K 31/12; A61K 31/352; A61K 31/375; A61K 31/4525; A61K 31/658; A61K 31/704; A61K 33/06; A61K 33/08; A61K 33/10; A61K 33/26

(57) ABSTRACT

The present invention relates to beverage compositions comprising natural products, particularly beverage compositions comprising a hibiscus extract and magnesium, and their use, for example, in reducing blood pressure, mitigating the progression of sub-clinical cardiovascular, metabolic and/or cardiovascular disorders, and the like.

### CROSS-REFERENCE TO RELATED APPLICATIONS

This application claims priority from U.S. Provisional Patent Application No. 63/490,299 filed 15 Mar. 2023, the entire contents of which are incorporated herein by reference.

### FIELD OF THE INVENTION

The present invention relates to beverage compositions comprising natural products. In particular, the present invention relates to beverage compositions comprising hibiscus and magnesium and their use in reducing blood pressure.

### BACKGROUND

Hypertension, or high blood pressure, is a high-priority risk factor for morbidity and mortality worldwide, leading to what is termed “premature death”. The prevalence of hypertension among adults in the United States is estimated at 50% or higher. Of particular concern is that roughly 20% of individuals with high blood pressure are unaware that they have this condition.

In 2017, the American College of Cardiology/American Heart Association (ACC/AHA) established new hypertension guidelines as a standard basis for medical practice. The guidelines define hypertension as a medical condition in which patients have a systolic blood pressure (SBP) of 130 mm Hg or higher and a diastolic blood pressure (DBP) of 80 mm Hg or higher. Normal blood pressure is defined as an SBP lower than 120 mm Hg with a DBP lower than 80 mm Hg.

Prehypertension is defined as having an SBP ranging from 120-129 mm Hg with a DBP higher than 80 mm Hg. Prehypertension is not recognized as a medical condition on its own; rather, it is a sub-clinical warning sign or health risk. As such, prehypertension is an indication that an individual has an increased risk of developing high blood pressure.

Prehypertension affects a large percentage of the population in the United States and worldwide. It is estimated that one third of American adults have prehypertension, and are typically unaware of it and its potential consequences in the long-term. In addition, 10-15% of American adolescents (12-19 years of age) have prehypertension or frank hypertension, which equates to upwards of 3 million children. Medical practitioners do not typically classify prehypertension as a diagnosable condition in persons who are free of relevant comorbidity, therefore there is no standard treatment. However, lifestyle modifications, including dietary changes, are recognized as beneficial in preventing or retarding the progression to frank cardiovascular disease (CVD).

Frequent consumption of sweetened drinks and other popular beverages (e.g., sodas, fruit juices, and energy drinks) significantly increases the risk of metabolic syndrome and CVD. In a recent study, the habitual consumption of sugar-sweetened beverages was found to worsen the risk of developing CVD, even among adults who exercise at above-average levels of physical activity. In addition, artificially-sweetened soft drinks are associated with premature deaths from circulatory diseases. Several studies in adolescents have shown that their high consumption of sweetened beverages is associated with increased ill effects, including cancer (e.g., rectal adenomas), obesity, cardiometabolic and other chronic diseases, in addition to decreased cognitive function and academic performance.

Therefore, alternative beverages and snacks that have heart-healthy properties and ingredients may benefit the overall well-being of millions of Americans and billions of adults and adolescents worldwide.

### SUMMARY OF THE INVENTION

In one aspect, the present invention provides a beverage composition comprising a hibiscus extract and magnesium.

In another aspect, the present invention provides a process for preparing a beverage composition of the invention, comprising combining a hibiscus extract and magnesium.

In another aspect, the present invention provides a beverage composition of the invention for reducing blood pressure or preventing an increase in blood pressure in a subject.

In another aspect, the present invention provides a beverage composition of the invention for preventing or reducing the risk of developing a disease or condition selected from the group consisting of cardiovascular disease (CVD), cancer (e.g., rectal adenomas), obesity, cardiometabolic disease, kidney stones, neuromuscular, cardiac or nervous disorders, stroke, coronary heart disease, heart failure, hyperlipidemia, hyperglycemia, blood fat, blood sugar, chronic kidney disease, atherosclerosis, endothelial dysfunction, and arterial stiffening, erectile dysfunction, premenstrual syndrome, preeclampsia, preterm birth, cerebral palsy, osteoporosis, asthma, chronic obstructive pulmonary disease (COPD), cystic fibrosis, coronary artery disease, myocardial infarction, arrhythmia, heart failure, vascular calcification, atopic dermatitis, psoriasis, cataract, glaucoma, hearing loss, liver cirrhosis, non-alcoholic fatty liber, hepatic cancer, insulin resistance, prediabetes and type 2 diabetes mellitus, or mitigating the progression of sub-clinical cardiovascular, metabolic and/or cardiovascular disorders, in a subject.

In another aspect, the present invention provides a beverage composition of the invention for reducing stress and/or anxiety, reducing muscle tension and/or cramps, reducing incidence of headaches and/or migraines, improving/elevating mood, improving sleep quality and/or reducing insomnia, improving cognitive function and/or academic performance, improving exercise performance and/or sexual performance, reducing dreaminess, inducing sleep, calming, elevating mood, reducing appetite, weight loss, and/or providing immune support, in a subject.

In yet another aspect, the present invention provides a method of reducing blood pressure or preventing an increase in blood pressure in a subject in need thereof comprising the subject consuming a beverage composition containing hibiscus and an effective amount of magnesium.

In yet another aspect, the present invention provides a method of preventing or reducing the risk of developing a disease or condition selected from the group consisting of cardiovascular disease (CVD), cancer (e.g., rectal adenomas), obesity, cardiometabolic disease, kidney stones, neuromuscular, cardiac or nervous disorders, stroke, coronary heart disease, heart failure, hyperlipidemia, hyperglycemia, blood fat, blood sugar, chronic kidney disease, atherosclerosis, endothelial dysfunction, and arterial stiffening, erectile dysfunction, premenstrual syndrome, preeclampsia, preterm birth, cerebral palsy, osteoporosis, asthma, chronic obstructive pulmonary disease (COPD), cystic fibrosis, coronary artery disease, myocardial infarction, arrhythmia, heart failure, vascular calcification, atopic dermatitis, psoriasis, cataract, glaucoma, hearing loss, liver cirrhosis, non-alcoholic fatty liber, hepatic cancer, insulin resistance, prediabetes and type 2 diabetes mellitus, or mitigating the progression of sub-clinical cardiovascular, metabolic and/or cardiovascular disorders, in a subject, the method comprising the subject consuming a beverage composition containing a hibiscus extract and an effective amount of magnesium.

In yet another aspect, the present invention provides a method of reducing stress and/or anxiety, reducing muscle tension and/or cramps, reducing incidence of headaches and/or migraines, improving/elevating mood, improving sleep quality and/or reducing insomnia, improving cognitive function and/or academic performance, improving exercise performance and/or sexual performance, reducing dreaminess, inducing sleep, calming, elevating mood, reducing appetite, weight loss, and/or providing immune support, in a subject, the method comprising the subject consuming a beverage composition containing a hibiscus extract and an effective amount of magnesium.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the beverage compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of values that fall within 10%, 9%, 8%, 7%, 6%, 5%, 4%, 3%, 2%, 1%, or less in either direction (greater than or less than) of the stated value unless otherwise stated or otherwise evident from the context (except where such number would exceed 100% of a possible value). In a particular embodiment, the term “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments may also be envisioned with replacement of the term “comprising” with the terms “consisting of” or “consisting essentially of.”

The phrase “pharmaceutically acceptable” is employed herein to refer to those agents of interest/compounds, salts, compositions, dosage forms, etc, which are—within the scope of sound medical judgment-suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “salts”, as used herein, embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts may be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid. The preparation of the pharmaceutically acceptable salts described above and other typical pharmaceutically acceptable salts is more fully described by Berge et al., “Pharmaceutical Salts,” J. Pharm. Sci., 1977:66:1-19.

The term “consumer” and “subject” are interchangeable and may be taken to mean any living organism which may be consuming the beverages of the present invention. As such, the terms “consumer” and “subject” may include, but are not limited to, any non-human mammal, primate, or human. In some embodiments, the “consumer” or “subject” is a mammal, such as mice, rats, other rodents, rabbits, dogs, cats, swine, cattle, sheep, horses, primates, or humans. In some embodiments, the patient or subject is an adult, child or infant. In some embodiments, the consumer or subject is a human.

As used herein, the term “substantially free” when used with respect to a component of a composition means that the component is absent from the composition or is present in the composition in a trace amount only, e.g., less than about 3% w/w of the total composition, including less than about 3%, 2.5%, 2%, 1.5%, 1%, 0.5%, 0.3%, 0.2%, 0.1%, 0.01 or 0% w/w based on the total weight of the composition.

As used herein, the term “alkyl” or “alkyl group” means a linear or branched, saturated hydrocarbon including one or more carbon atoms (e.g., one, two, three, four, five, six, seven, eight, nine, ten, or more carbon atoms), which is optionally substituted. The notation “C₁-C₁₀ alkyl” means an optionally substituted linear or branched, saturated hydrocarbon including 1-10 carbon atoms, “C₁-C₆ alkyl” means an optionally substituted linear or branched, saturated hydrocarbon including 1-6 carbon atoms, “C₁-C₃ alkyl” means an optionally substituted linear or branched, saturated hydrocarbon including 1-3 carbon atoms, and the like. Unless otherwise specified, an alkyl group described herein refers to both unsubstituted and substituted alkyl groups. Examples of suitable alkyl groups may include, but are not limited to, methyl, ethyl, 1-propyl, isopropyl, 1-butyl, 2-butyl, isobutyl, sec-butyl, tert-butyl, pentyl, hexyl, heptyl, octyl, nonyl, decyl, and the like.

As used herein, the term “alkenyl” or “alkenyl group” means a linear or branched hydrocarbon including two or more carbon atoms (e.g., two, three, four, five, six, seven, eight, nine, =or more carbon atoms) and at least one double bond, which is optionally substituted. Unless indicated otherwise, the stereochemistry about each double bond may be independently cis or trans, or E or Z, as appropriate. The notation “C₂-C₁₀ alkenyl” means an optionally substituted linear or branched hydrocarbon including 2-10 carbon atoms and at least one carbon-carbon double bond, “C₂-C₆ alkenyl” means an optionally substituted linear or branched hydrocarbon including 2-6 carbon atoms and at least one carbon-carbon double bond, “C₂-C₃ alkenyl” means an optionally substituted linear or branched hydrocarbon including 2-3 carbon atoms and at least one carbon-carbon double bond, and the like. An alkenyl group may include one, two, three, four, or more carbon-carbon double bonds. For example, C₁₀ alkenyl may include one or more double bonds. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, or more carbon atoms) and at least one carbon-carbon triple bond, which is optionally substituted. The notation “C₂-C₁₀ alkynyl” means an optionally substituted linear or branched hydrocarbon including 2-10 carbon atoms and at least one carbon-carbon triple bond, “C₂-C₆ alkynyl” means an optionally substituted linear or branched hydrocarbon including 2-6 carbon atoms and at least one carbon-carbon triple bond. An alkynyl group may include one, two, three, four, or more carbon-carbon triple bonds. For example, C₁₀ alkynyl may include one or more carbon-carbon triple bonds. Unless otherwise specified, an alkynyl group described herein refers to both unsubstituted and substituted alkynyl groups.

As used herein, the term “alkoxy” or “alkoxyl group” means a chemical substituent of formula-OR, where R is an alkyl group as defined herein (e.g., O—C₁-C₁₀ alkyl, O—C₁-6 alkyl or O—C₁-C₃ alkyl), unless otherwise specified. Exemplary alkoxy groups include methoxy, ethoxy, propoxy (e.g., n-propoxy and isopropoxy), t-butoxy, and the like.

As used herein, the term “aryl” refers to an optionally substituted monocyclic, or fused polycyclic, aromatic carbocycle (i.e., a ring structure having ring atoms that are all carbon). The aryl group may have from 6-10 atoms per ring, denoted C₆-10 aryl. Examples of suitable aryl groups may include, but are not limited to, phenyl, naphthyl, phenanthryl. As used herein, the term “aryl” is also intended to encompass optionally substituted partially saturated bicyclic aromatic carbocyclic moiety in which a phenyl and a cycloalkyl or cycloalkenyl group are fused together to form a cyclic structure, such as tetrahydronaphthyl, indenyl or indanyl. The aryl group may be a terminal group or a bridging group.

As used herein, the term “aryloxy” or “aryloxyl group” means a chemical substituent of formula-OR, where R is an aryl group as defined herein (e.g., O—C₆-C₁₀ aryl), unless otherwise specified.

As used herein, the term “arylalkyl” or “arylalkyl group” means a chemical substituent of formula —R^{a}-R^{b}, where R^{a} is an alkyl group as defined herein and R^{b} is an aryl group as defined herein (e.g., —C₁-C₁₀ alkyl-C₁-C₁₀ aryl), unless otherwise specified.

As used herein, the term “amine” means a chemical substituent of formula —NR^{c}R^{d}, wherein R^{c} and R^{d} are each independently an alkyl group as defined herein (e.g., N(C₁-C₁₀ alkyl)(C₁-C₁₀ alkyl)), unless otherwise specified. Exemplary amine groups include, dimethylamine, diethylamine, diisopropylamine, and the like.

As used herein, the term “amino acid” refers to any natural or non-natural amino acid, including but not limited to: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, homoserine, and norleucine. As used herein, the term “amino acid ester” refers to any amino acid in which the hydrogen atom of the acidic hydroxyl group of that acid is replaced by an “alkyl” group as defined elsewhere herein.

A reference to a percentage (%) content throughout this specification is to be taken to mean a percentage by weight, denoted “% w/w” or “wt. %”. Unless otherwise specified, any reference to a percentage by weight of a component of a composition or formulation described herein refers to the percentage by weight of the specified component with respect to the total components of the composition or formulation. Certain components of the compositions described herein may be provided as a liquid. In such cases, it may be more convenient to measure the amount of a liquid component as a volume (e.g., in mL). A skilled person will be able to readily determine the weight of a liquid component given its volume by multiplying the volume (in mL) by the density of the liquid (in g/mL), for example, as measured at 25° C.

The terms “composition” and “formulation” are used interchangeably throughout this specification and have the same meaning.

By hereby reserving the right to proviso out or exclude any individual members of any such group, including any sub-ranges or combinations of sub-ranges within the group, that may be claimed according to a range or in any similar manner, less than the full measure of this disclosure may be claimed for any reason. Further, by hereby reserving the right to proviso out or exclude any individual substituents, analogs, compounds, ligands, structures, or groups thereof, or any members of a claimed group, less than the full measure of this disclosure may be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

The reference to any prior art in this specification is not, and should not be taken as, an acknowledgment or any form of suggestion that that prior art forms part of the common general knowledge.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

As used herein, the term “beverage composition” encompasses liquid beverage compositions as well as solid (dry) compositions that, when combined with an aqueous liquid provide a beverage suitable for human consumption. Thus, various embodiments of the invention are directed to drink mixes (e.g., liquid and dry mixes), ready-to-drink beverages, energy drinks, and other orally consumable liquids and components thereof, collectively “beverage compositions”, containing combinations of ingredients capable of providing an effect, such as a soothing effect, on the consumer. Such effects may include, but are not limited to, reducing blood pressure, reducing blood fat, reducing blood sugar, reducing stress, reducing insomnia, reducing dreaminess, reducing hypertension, reducing hyperlipidemia, reducing hyperglycemia, supporting restful sleep (e.g., inducing sleep), calming, supporting healthy mood (e.g., elevating mood), and, in some embodiments, reducing appetite, and the like and combinations thereof.

The present invention relates to beverage compositions comprising natural products, particularly a hibiscus extract (e.g., a hibiscus leaf, root and/or flower extract) and magnesium. Such compositions may provide safe and effective natural beverages that aid in reducing blood pressure, which may provide short- and/or long-term health benefits to the cardiovascular system, as well as other bodily organs and tissues of mammals, including humans.

The beverages of the present invention comprise a plant extract (e.g., a plant leaf, root and/or flower extract). In some embodiments, the beverage is a tea and/or tisane comprising a plant extract. The plant extract may include, but is not limited to, extracts of resin, sap, latex, raw, fresh, dried, freeze-dried, fermented, or unfermented leaves, roots, flowers or other parts of plants such as, for example, oolong tea, iron kannon, color species, golden katsura, wushuiwa tea, red tea, white tea, black tea, darjeeling, assam, Sri Lanka camellia, Ginkgo biloba leaves, bamboo leaves, loquat leaves, mulberry leaves, wolfberry leaves, bamboo leaves, komatsuna, rooibos, kumazasa, dokudami, amacha eel, honeysuckle, primrose, Kakidooshi, Kawaraketsumci, Gymnema sylvestre, Garcinia cambogia, Glycyrrhiza glabra, Passiflora incarnata, Valeriana officinalis, Punica granatum Magnolia officinalis, Crataegus monogyna Jacq. (common and related Hawthorn species), Hypericum perforatum (St John's wort), Euterpe oleracea (açaí), Elettaria cardamomum (cardamom) Crocus sativus L. (saffron), Boswellia sacra (also known as Boswellia carteri, the frankincense tree, or the olibanum tree), Lavandula angustifolia and/or Lavandula latifolia (and/or other lavender species and varieties), twilight tea (walnut family), persimmon tea (rose family), kidachi aloc, chamomile, hibiscus, peppermint, thyme, lemongrass, lemon peel, lemon balm, rosehip, rosemary, celery, cavender, calendula, carrot, cucumber, yerba mate, coca extract, ginger, chlorophyll, aloe, cinnamon, ginseng, and the like, and combinations thereof.

In some embodiments, the plant extract may be a hibiscus extract (e.g., a hibiscus leaf and/or flower extract). The hibiscus extract may be an extract from any plant of the Hibiscus species, including but not limited to Hibiscus sabdariffa or Hibiscus acetosella. For example, the hibiscus extract may be a “hibiscus tea”. Hibiscus tea is a tisane (also known as an herbal tea) commonly made from hibiscus flowers, primarily including, but not limited to the sepals of Hibiscus sabdariffa or the petals of Hibiscus acetosella; however, embodiments of the present invention may encompass other species and varieties of hibiscus. Extracts of Hibiscus species may contain safe and effective concentrations of bioactive elements and compounds, including, but not limited to, minerals, vitamins (including, but not limited to niacin, riboflavin, ascorbic acid, α- and β-tocopherols), amino acids, ascorbic acid, and other organic acids, lipids, polysaccharides and/or oligosaccharides, secondary metabolites, alkaloids, polycyclic aromatic organic compounds, quinones, terpenes and/or terpenoids, phytosteroids, phenolic, flavonoid compounds, anthocyanin compounds, sambubioside compounds (including, but not limited to delphinidin-3-O-sambubioside and cyanidin-3-O-sambubioside), quercetin, luteolin and its glycosides, chlorogenic acid, gossypetin, epigallocatechin gallate, and hibiscetin.

An “extract” as used herein refers to a composition containing one or more compounds found in a plant, particularly the plant leaves or flowers. In some embodiments, the extract is an aqueous extract. Aqueous extracts may be prepared, for example, by steeping the plant leaves or flowers in a liquid, for example, an aqueous liquid such as water (e.g., purified water, natural or spring water), hydroalcoholic mixture (e.g., an ethanol/water solution), fruit or vegetable juice, an emulsion (e.g., an animal milk or plant-based milk or a smoothie), to transfer various water soluble compounds in the plant leaves or flowers to the liquid. In some embodiments, the liquid may be heated to facilitate transfer of the water soluble compounds into solution. The remaining plant leaves or flowers may be removed from the liquid composition (e.g., by straining or filtering), or they may be retained in the liquid composition. In some embodiments, the extract is a tea. As used herein, a “tea” refers to a beverage made by infusing the dried crushed leaves of a tea plant in hot (preferably boiling) water. In some embodiments, the extract may be prepared from plant leaves or flowers that have undergone a process to increase the concentration of these compounds beyond that which would be expected to be produced by, for example, steeping the flowers in a liquid. For example, in some embodiments, an extract may be created by steeping the plant leaves or flowers in a liquid and selectively removing the liquid or a portion of the liquid leaving the compounds from the plant leaves or flowers. In other embodiments, the compounds from the plant leaves or flowers may be separated using, for example, chromatography. In other embodiments, the extract may be diluted with an aqueous liquid to achieve the desired volume and/or concentration of components. In the case of hibiscus, a hibiscus extract may include, for example, hydroxycitric acid (1,2-dihydroxypropane-1,2,3-tricarboxylic acid), a derivative of citric acid found in a variety of tropical plants including Garcinia cambogia and Hibiscus subdariffa. Other compounds derived from the plant leaves or flowers may also be present in such extracts, including anthocyanins such as delphinidin-3-O-sambubioside (Dp-samb) and cyanidin-3-O-sambubioside (Cy-samb).

Certain plant extracts may be associated with one or more beneficial effects. For example, hibiscus tea has been associated with blood pressure reducing, blood fat reducing, blood sugar reducing, stress reducing, insomnia reducing, dreaminess reducing, hypertension reducing, hyperlipidemia reducing, hyperglycemia reducing, sleep inducing, calming, mood elevating, and/or appetite-reducing effects. Other advantageous effects associated with hibiscus may include weight loss effects, immune support and kidney stone prevention. Hibiscus tea is also substantially free of caffeine and calories (including being substantially free of sugar, fat and carbohydrates).

The beverages of the present invention also comprise a mineral. Such minerals may include, but are not limited to, boron, phosphorus, titanium, chromium, magnesium, manganese, iron, cobalt, nickel, copper, zinc, molybdenum, cadmium, aluminum, and the like and combinations thereof. In some embodiments, such minerals may simulate a taste profile of mineral water from a specific region. For example, the mineral ingredients may simulate a taste profile of water from the German Alps, the Italian Alps, or the French Alps. A skilled person will readily be able to adjust the mineral profile of a beverage composition disclosed herein to achieve the desired properties. In some embodiments, the minerals may impact the effect of the beverage on the consumer. For example, in some embodiments, the mineral may enhance the blood pressure reducing, blood fat reducing, blood sugar reducing, stress reducing, insomnia reducing, dreaminess reducing, hypertension reducing, hyperlipidemia reducing, hyperglycemia reducing, sleep inducing, calming, mood elevating, and/or appetite-reducing effect of the plant extract. The mineral, or combination thereof, may be present in the beverage compositions disclosed herein in an amount effective to achieve a particular taste profile and/or to enhance the effects of the plant extract. Additionally or alternatively, the mineral may provide one or more beneficial effects that may be enhanced by the plant extract. Such effects may depend on the specific mineral any will be apparent to those skilled in the art. For example, magnesium may provide one or more of the beneficial effects described elsewhere herein. The amount of a particular mineral that may be present in the beverage compositions of the present invention may vary depending on the mineral. A skilled person will be able to select a suitable amount depending on the taste profile and/or effects to be achieved, in view of such factors as the recommended daily intake of the specific mineral.

In some embodiments, the mineral present in the beverage compositions of the present invention is magnesium. As used herein, the term “magnesium” refers to magnesium cations (Mg²⁺), which may be provided in any suitable salt form that is recognized as safe for human consumption, preferably which have been granted General Recognized as Safe (GRAS) status by the United States Food and Drug Administration (FDA). Forms of magnesium (Mg²⁺) that have been granted GRAS status include, but are not limited to magnesium carbonate, magnesium chloride, magnesium hydroxide, magnesium oxide, magnesium phosphate, magnesium stearate and magnesium sulfate. In some embodiments, the magnesium is in the form of magnesium ascorbate, magnesium citrate or magnesium taurate. In a particular embodiment, the magnesium (Mg²⁺) is provided as an organic salt, such as magnesium ascorbate, magnesium carbonate, magnesium citrate or magnesium taurate, which may have greater bioavailability than inorganic magnesium salts. Combinations of two or more magnesium salts are also contemplated by the present invention.

Magnesium is an essential mineral involved in numerous metabolic processes. An imbalance in magnesium levels in the body can lead to neuromuscular, cardiac or nervous disorders, which may include symptoms such as neuromuscular dysfunction, muscle weakness, and muscle cramping, among others. Magnesium is also recognized for its ability to reduce the risk of high blood pressure (hypertension). Hypertension is a risk factor for a number of chronic diseases including, but not limited to, stroke, coronary heart disease, heart failure and chronic kidney disease. Magnesium supplementation has also been shown to significantly lower blood pressure (systolic blood pressure and/or diastolic blood pressure) in individuals with insulin resistance, prediabetes and type 2 diabetes, which may reduce or alleviate one or more of the systems associated with these diseases. With more than one third of American adults estimated to have prediabetes, and the prevalence of insulin resistance ranging from about 15-50% of adults worldwide, the beverage compositions of the present invention have the potential for significant impact on global public health. Other benefits of lowering blood pressure may include, but are not limited to, reduced stress and muscle tension, reduced incidence headaches, improved/elevated mood, improved sleep quality, some of which are also recognized as risk factors for the development of cardiovascular disease.

A single serving of a beverage composition of the present invention may comprise an amount of magnesium sufficient to provide a desired daily dose of magnesium to a consumer, such as the Recommended Dietary Allowance (RDA), or an amount which, when combined with other sources of magnesium in the diet of the consumer equates to the RDA of magnesium. For example, for adults 19 years of age and older, the Recommended Dietary Allowance for magnesium intake is 400-420 mg/day for men and 310-320 mg/day for women. Intake recommendations for magnesium, including the RDA, are provided in the Dietary Reference Intakes developed by the Food and Nutrition Board at the Institute of Medicine of the National Academies (Institute of Medicine (IOM). Food and Nutrition Board. Dietary Reference Intakes: Calcium, Phosphorus, Magnesium, Vitamin D and Fluoride. Washington, DC: National Academy Press, 1997), the entire contents of which are incorporated herein by reference. The RDAs for magnesium are provided in Table 1 below.

TABLE 1
Recommended Dietary Allowances for magnesium

| Age | | Male | Female | Pregnancy | Lactation |
|---|---|---|---|---|---|
| Birth to 6 months | | 30 mg* | 30 mg* | | |
| 7-12 | months | 75 mg* | 75 mg* | | |
| 1-3 | years | 80 mg | 80 mg | | |
| 4-8 | years | 130 mg | 130 mg | | |
| 9-13 | years | 240 mg | 240 mg | | |
| 14-18 | years | 410 mg | 360 mg | 400 mg | 360 mg |
| 19-30 | years | 400 mg | 310 mg | 350 mg | 310 mg |
| 31-50 | years | 420 mg | 320 mg | 360 mg | 320 mg |
| 51+ | years | 420 mg | 320 mg | | |

*Adequate Intake (AI)

In some embodiments, a single serving of the beverage composition may comprise magnesium in an amount of from about 30 mg to about 500 mg, or about 30 mg to about 420 mg, or about 30 mg to about 400 mg, or about 30 mg to about 320 mg, or about 30 mg to about 310 mg, or about 80 mg to about 500 mg, or about 80 mg to about 420 mg, or about 80 mg to about 400 mg, or about 80 mg to about 320 mg, or about 80 mg to about 310 mg, or any subrange thereof. For example, the magnesium may be present in a single serving of the beverage composition in an amount of 30 mg, 75 mg, 80 mg, 130 mg, 240 mg, 310 mg, 320 mg, 350 mg, 360 mg, 400 mg, 410 mg, 420 mg, or 500 mg per serving. In an embodiment, the magnesium may be present in the beverage composition in an amount of from about 310 mg to about 420 mg per serving. A skilled person will be able to determine a suitable amount of a particular magnesium salt to provide the desired amount of magnesium in the beverage composition. In an embodiment, the magnesium may be present in the beverages of the present invention at or below the RDA. Depending on the amount of magnesium in the beverage composition, the beverage composition may be consumed one, two, three, four or more times per day, the combined total of which equates to a single serving of magnesium (taking into account other sources of magnesium that may present in the diet of the consumer).

In a particular embodiment, the present invention provides a beverage composition comprising a hibiscus extract and magnesium. Hibiscus is rich in anthocyanins, which are a class of water-soluble flavonoids that are responsible for the deep red/magenta color of hibiscus. Without wishing to be bound by theory, it is postulated that the anthocyanins present in aqueous hibiscus extracts form Mg-organoanthocyanin complexes that increase the bioavailability of the magnesium. Thus, the combination of hibiscus and magnesium may enhance one or more of the beneficial effects of magnesium, such as reducing blood pressure. By enhancing the beneficial effects of magnesium, the presence of hibiscus extracts in the beverages of the present invention may reduce the amount of magnesium required to achieve one or more of the beneficial effects (e.g., blood pressure lowering effects) of the magnesium relative to the consumption of magnesium alone. This may be advantageous, for example, by reducing the incidence of adverse effects that may be associated with consuming magnesium, including but not limited to diarrhea and vomiting.

Thus, it is to be understood that the hibiscus extracts present in the beverage compositions of the present invention comprise anthocyanins, such as delphinidin-3-O-sambubioside (Dp-samb) and cyanidin-3-O-sambubioside (Cy-samb). Other anthocyanins may also be present in the extracts of the present invention. The amount of anthocyanins present in the beverage composition may be sufficient such that at least a portion of the magnesium present in the composition is complexed with the anthocyanins. For example, the molar ratio of anthocyanins to magnesium may be in the range of from about 20:1 to about 1:20, or from about 10:1 to about 1:10, or from about 5:1 to about 1:5, or from about 2:1 to about 1:2.

Further, it is well known to those skilled in the art that angiotensin-converting enzyme (ACE) plays a critical role in the regulation of arterial blood pressure. Several flavonoids found in hibiscus, such as quercetin, luteolin, epigallocatechin gallate, chlorogenic acid, apigenin, anthocyanins (specifically delphinidin and cyanidin compounds) as well as the glycyrrhizin as present in Glycyrrhiza glabra (licorice) demonstrate inhibition of ACE activity. ACE inhibitors have also been shown to promote magnesium conservation within the body. Thus, in some embodiments, the beverage compositions disclosed herein further comprise licorice (e.g., licorice root). The presence of ACE inhibitors in the beverage compositions of the present invention may further contribute to blood pressure-lowering and other heart-healthy effects.

Preferably, the beverage compositions of the present invention are low in sodium, which is associated with high blood pressure. In an embodiment, a single serving of the beverage composition contains less than 140 mg sodium, or less than 100 mg sodium, or less than 70 mg sodium, or less than 50 mg sodium, or less than 35 mg sodium.

In some embodiments, the beverages of the present invention may further comprise one or more additional active components, such as an anti-inflammatory agent.

In some embodiments, the anti-inflammatory component may be, for example, a cannabinoid. In addition to their anti-inflammatory properties, cannabinoids may provide other beneficial effects when used in the beverage compositions of the present invention, such as promoting relaxation, reducing stress and/or anxiety, improved sleep quality and/or alleviating pain. “Cannabinoid” refers to a compound that is capable of acting on cannabinoid receptors (e.g., interacting with or binding to) cannabinoid receptors (including CB1 and CB2 receptors, or any endocannabinoid receptor, including endocannabinoid receptor(s) yet to be discovered). A skilled person may readily determine whether a compound interacts with cannabinoid receptor using routine screening assays, such as the ligand binding assays described by Devane et al. (1988) Mol Pharmacol 34:605-613 or by in silico prediction of CB1 affinity (see, e.g., Paulke et al. (2016) Toxicoly Letters 245:1-6). In some embodiments, the cannabinoid is an isolated cannabinoid. As used herein the term “isolated” means a single (individual) target cannabinoid, e.g., a target cannabinoid that is isolated or substantially free of other (non-target) cannabinoids and/or active agents, including plant-isolated cannabinoids. The isolated cannabinoid may have a purity of greater than about 80%, 90%, 95% or 98% by weight. In a particular embodiment, the isolated cannabinoid is a food grade cannabinoid (>95% purity) or a pharmaceutical grade (>98% purity).

Cannabinoids may be produced naturally in the body (endocannabinoids), found in cannabis and some other plants (phytocannabinoids), or manufactured artificially (synthetic cannabinoids). Cannabinoids produced by cannabis or hemp plants during cultivation and growth (phytocannabinoids) include tetrahydrocannabinolic acid (THCA), cannabinolic acid (CBNA), cannabidiolic acid (CBDA), cannabichromenic acid (CBCA), and cannabigerolic acid (CBGA). In some embodiments, such cannabinoids may be decarboxylated moieties to produce tetrahydrocannabinol (THC), cannabinol (CBN), cannabidiol (CBD), cannabinchromene (CBC) and cannabigerol (CBG). Cannabinoids also encompass the metabolites of these and other cannabinoids that result upon oral consumption of these compounds by humans, such as, but not limited to, 11-OH-THC and THC COOH (also known as 11-nor-9-carboxyTHC).

Cannabinoids suitable for use in the present invention may be synthesized by chemical or biological methods. In some exemplary beverage compositions, the cannabinoids may be derived (isolated) from plants including hemp, Echinacea purpurea, Echinacea angustifolia, Acmella oleracea, Helichrysum umbraculigerum, Radula marginata, and combinations thereof and/or oils made from these plants. In other examples, the cannabinoids may be selected from cannabinoids that are manufactured, chemically synthesized, or combinations thereof.

In some embodiments, the cannabinoid may be, for example, a synthetic cannabinoid or cannabidiol such as, for example, Δ-5-cannabidiol (2-(6-isopropenyl-3-methyl-5-cyclohexen-1-yl)-5-pentyl-1,3-benzenediol); Δ-4-cannabidiol (2-(6-isopropenyl-3-methyl-4-cyclohexen-1-yl)-5-pentyl-1,3-benzenediol); Δ-3-cannabidiol (2-(6-isopropenyl-3-methyl-3-cyclohexen-1-yl)-5-pentyl-1,3-benzenediol); Δ-3′7-cannabidiol (2-(6-isopropenyl-3-methylenecyclohex-1-yl)-5-pentyl-1,3-benzenediol); Δ-2-cannabidiol (2-(6-isopropenyl-3-methyl-2-cyclohexen-1-yl)-5-pentyl-1,3-benzenediol); Δ-1-cannabidiol (2-(6-isopropenyl-3-methyl-1-cyclohexen-1-yl)-5-pentyl-1,3-benzenediol); Δ-6-cannabidiol (2-(6-isopropenyl-3-methyl-6-cyclohexen-1-yl)-5-pentyl-1,3-benzenediol), Δ-9-tetrahydrocannabinol (Δ-9-THC), HU-210 (6aR,10aR)-9-(hydroxymethyl)-6,6-dimethyl-3-(2-methyloctan-2-yl)-6H,6aH,7H, 10H, 10aH-benz o[c]ochromen-1-ol), cannabidivarin (CBDV), cannabichromene (CBC), cannabichromevarin (CBCV), cannabigerol (CBG), cannabigerovarin (CBGV), cannabielsoin (CBE),cannabicyclol (CBL),cannabivarin (CBV), and cannabitriol (CBT), tetrahydrocannibivarin (THCV), cannabigerol monomethyl ether (CBGM), cannabichromenic acid (CBCA), Δ-1-tetrahydrocannabinolic acid (THCA), and cannabidiolic acid (CBDA), rimonabant, JWH-018 (naphthalen-1-yl-(1-pentylindol-3-yl)methanone), JWH-073 (naphthalen-1-yl-(1-butylindol-3-yl)methanone), CP-55940 (2-[(1R,2R,5R)-5-hydroxy-2-(3-hydroxypropyl)cyclohexyl]-5-(2-methyloctan-2-yl)phenol), dimethylheptylpyran, HU-331 (3-hydroxy-241R)-6-isopropenyl-3-methyl-cyclohex-2-cn-1-yl]-5-pentyl-1,4-benzoquinone), SR144528 (5-(4-chloro-3-methylphenyl)-1-[(4-methylphenyl)methyl]-N—R1S,2S,4R)-1,3,3-trimethylbicyclo[2.2.1]heptan-2-yl]-1H-pyrazole-3-carboxamide), WIN 55,212-2 ((11R)-2-methyl-11-[(morpholin-4-yl)methyl]-3-(naphthalene-1-carbonyl)-9-oxa-1-azatricyclo [6.3.1.04,12]dodeca-2,4(12),5,7-tetraene), JWH-133 ((6aR,10aR)-3-(1,1-dimethylbutyl)-6a, 7,10,10a-tetrahydro-6,6,9-trimethyl-6H-dibenzo[b,d]pyran), levonatradol, and AM-2201 (1-[(5-fluoropentyl)-1H-indol-3-yl]-(naphthalen-1-yl)methanone), Δ-8-tetrahydrocannabinol (Δ-8-THC), 11-hydroxy-Δ-9-tetrahydrocannabinol, Δ-11-tetrahydrocannabinol, 11-hydroxy-tetracannabinol, and the like and combinations thereof.

In an embodiment, the cannabinoid is a tetrahydropyran analog, such as, Δ-9-tetrahydrocannabinol, Δ-8-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1hydroxy-6,6-dimythel-9H-dibezo[b,d]pyran-9-ol, (−)-(3S,4S)-7-hydroxy-delta-6-tetrahydrocannabinol-1,1-dimethylheptyl, (+)-(3S,4S)-7-hydroxy-Δ-6-tetrahydrocannabinol, and Δ-8-tetrahydrocannabinol-11-oic acid. In an embodiment, the cannabinoid is a piperidine analog, such as, (−)-(6S,6aR,9R,10aR)-5,6,6a,7,8,9,10,10a-octahydro-6-methyl-1-3-[(R)-1-methyl-4-phenylbutoxy]-1,9 phenanthridinediol 1-acetate). In an embodiment, the cannabinoid is an aminoalkylindole analog, such as, (R)-(+)-[2,3-dihydro-5-methyl-3-(4-morpholinylm-ethyl)-pyrrolo[1,2,3,-de]-1,4-benzoxazin-6-yl]-1-naphthelenyl-methanone. In an embodiment, the cannabinoid is an open pyran-ring analog, such as, 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3′,4′,5′,6′-hexahydrobiphenyl, lipophilic alkylamides, such as, dodeca-2E,4E,8Z,10E/Z-tetraenoic-acid-isobutylamide.

In certain embodiments, the cannabinoid may be a compound of Formula I:

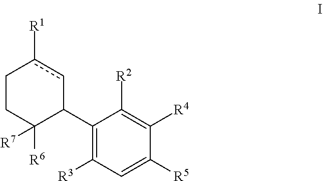

where R¹ is selected from hydrogen, hydroxyl, halogen (F, Cl, Br, I), methyl, linear or branched C₂-C₁₀ alkyl, linear or branched C₂-C₁₀ alkenyl, linear or branched C₂-C₁₀ substituted alkyl, and linear or branched C₂-C₁₀ substituted alkenyl, R² and R³ are each independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is selected from hydrogen, hydroxyl, halogen, substituted or unsubstituted alkyl, carboxyl, alkoxy, aryl, aryloxy, arylalkyl, R⁵ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, and R⁶ and R⁷ are each independently selected from hydrogen, hydroxyl, halogen, substituted or unsubstituted alkyl, substituted or unsubstituted alkenyl, substituted or unsubstituted alkynyl, carboxyl, alkoxy, aryl, aryloxy, arylalkyl, amino. The cycloalkyl moiety may or may not include double bonds such as the optional double bond indicated by the dashed line. The compounds of Formula I encompass any stereoisomers, salts, and solvates thereof.

In particular embodiments of Formula I, R¹, R², R³, R⁴, R⁵, R⁶ and R⁷ may each be a functional group, pro-functional group, or isostere thereof, or may be metabolized to an active functional group. In some embodiments, at least R¹, R², R⁴, and R⁵ may be hydrophobic. For example, each of R¹, R², R⁴, and R⁵ maybe independently selected from hydrogen, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. In some embodiments, R¹ and R³ may be a functional group capable of making a hydrogen bond with a functional group associated with the binding pocket of a RAS protein. For example, R¹ and R³ may be independently selected from hydrogen, hydroxyl, carboxyl, amino, alkyloxy, aryloxy, ketonyl, ester, or ether.

In certain embodiments, the cannabinoid may be a compound of Formula II:

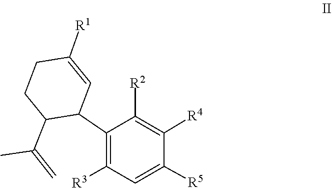

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is hydrogen, hydroxyl, halogen, substituted or unsubstituted alkyl, carboxyl, alkoxy, aryl, aryloxy, arylalkyl, and R⁵ is independently selected from hydrogen, hydroxyl, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. The compounds of Formula II encompass any stereoisomers, salts, and solvates thereof.

In particular embodiments of Formula II, R¹, R², R³, R⁴ and R⁵ may each be a functional group, pro-functional group, or isostere thereof, or may be metabolized to an active functional group. In some embodiments, at least R¹, R², R⁴, and R⁵ may be hydrophobic. For example, each of R¹, R², R⁴, and R⁵ be independently selected from hydrogen, halogen, methyl, linear or branched C₂-C₁₀ alkyl, linear or branched C₂-C₁₀ alkenyl, linear or branched C₂-C₁₀ substituted alkyl, or linear or branched C₂-C₁₀ substituted alkenyl. In some embodiments, R¹ and R³ may be a functional group capable of making a hydrogen bond with a functional group associated with the binding pocket of a RAS protein. For example, R¹ and R³ may be independently selected from hydrogen, hydroxyl, carboxyl, amino, alkyloxy, aryloxy, ketonyl, ester, or ether.

In some embodiments, R⁵ may be linear or branched C₂-C₁₀ alkyl. Thus, in certain embodiments, the cannabinoid may be a compound of Formula III:

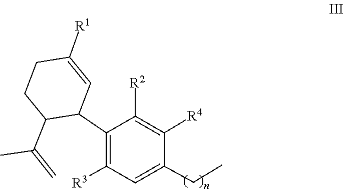

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is selected from hydrogen, substituted or unsubstituted alkyl, carboxyl, alkoxy, aryl, aryloxy, arylalkyl, halo or amino, R² and R³ are independently selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, and n may an integer of 2 to 10 and the like. The compounds of Formula III encompass stereoisomers, salts, and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof.

In some embodiments, the cannabinoid may be a compound of Formula IV:

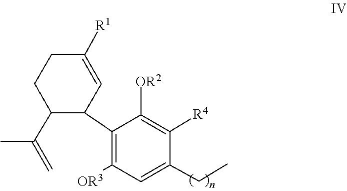

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are independently selected from hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is selected from hydrogen, substituted or unsubstituted alkyl, carboxyl, alkoxy, aryl, aryloxy, arylalkyl, halo or amino, and n may an integer of 2 to 10 and the like. The compounds of Formula IV encompass stereoisomers, salts, and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof.

In some embodiments, R⁶ or R⁷ as illustrated in Formula I may be covalently connected to R³ to produce a cyclized moiety. For example, in certain embodiments, the cannabinoid may be a compound of Formula V:

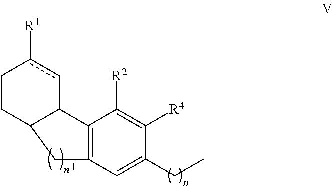

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² is selected from hydrogen, hydroxyl, halogen, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ alkyl ether, linear or branched C₂-C₁₀ substituted alkyl ether, linear or branched C₂-C₁₀ alkyl ester, linear or branched C₂-C₁₀ substituted alkyl ester, linear or branched C₂-C₁₀ acyl, linear or branched C₂-C₁₀ substituted acyl, amine or amino acid, amino acid ester, R⁴ is selected from hydrogen, substituted or unsubstituted alkyl, carboxyl, alkoxy, aryl, aryloxy, arylalkyl, halo or amino, and n and n¹ are each, individually, an integer of 2 to 10 and the like. The compounds of Formula V encompass stereoisomers, salts, and solvates thereof. In some embodiments, R² may be a linear or branched, substituted or unsubstituted C₂-C₁₀ acyl having a carboxylic acid terminus thereby producing a dicarboxylic acid, and salts thereof.

In some embodiments, R⁶ or R⁷ as illustrated in Formula I may be covalently connected to R³ to produce a cyclized moiety. For example, in certain embodiments, the cannabinoid may be a compound of Formula VI:

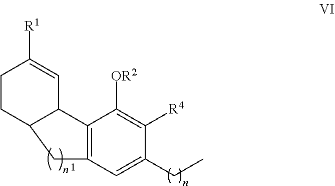

where R¹ is selected from hydrogen, hydroxyl, halogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² is selected from hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is selected from hydrogen, substituted or unsubstituted alkyl, carboxyl, alkoxy, aryl, aryloxy, arylalkyl, halo or amino. In various embodiments, n and n¹ are each, individually, an integer of 2 to 10 and the like. In some embodiments, the aliphatic chains created by n and n¹ may include one or more heteroatoms such as oxygen, nitrogen, sulfur. Additionally, a heteroatom may occupy the position at which the heterocycle is attached to the remainder of the molecule. The compounds of Formula VI encompass stereoisomers, salts, and solvates thereof. In some embodiments, R² may be a linear or branched, substituted or unsubstituted C₂-C₁₀ alkyl having a carboxylic acid terminus thereby producing a dicarboxylic acid, and salts thereof.

In some embodiments, the cannabinoid may be a mimetic, salt, solvate, metabolite, metabolic precursor, prodrug of any of the aforementioned compounds, and combinations thereof. The term “prodrug” is used in its broadest sense and encompasses those derivatives that are converted in vivo to the active agent (i.e., a target cannabinoid). Such derivatives would readily occur to those skilled in the art.

Unless otherwise stated, structures depicted above are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

The cannabinoid, such as the exemplary cannabinoids that are identified above, may be present in a beverage composition as described herein in any suitable form. For example, some exemplary beverage compositions may contain an individual cannabinoid stereoisomer, whereas some exemplary beverage compositions may contain a combination of cannabinoid stereoisomers. For example, in some exemplary beverage compositions, the cannabinoid may be cannabidiol (2-(6-isopropenyl-3-methyl-5-cyclohexen-1-yl)-5-pentyl-1,3-benzenediol) or any isomer or stereoisomer of cannabidiol, which has 7 double bonds and 30 stercoisomers; all of the foregoing may be suitable for use in the beverage compositions of the present invention. Similarly, some exemplary beverage compositions may comprise the various isomers and stereoisomers of cannabidiol (CBD), cannabigerol (CBG), cannabinol (CBN), cannabichromene (CBC), 49-tetrahydrocannabivarin (THCV), and cannabivarin (CBV), cannabidivarin (CBDV), cannabinodiol (CBND), cannabielsion (CBE), cannabicyclol (CBL), cannabitriol (CBT), cannabidolic acid (CBDA) and/or other cannabinoids not explicitly identified.

In some exemplary beverage compositions, the cannabinoids present in the compositions may be cannabinoid analogs. The term “cannabinoid analogs” refers to synthetically produced compounds that are structurally similar, but not structurally identical, to a target cannabinoid. A skilled person can readily determine whether a compound that is structurally similar, but not structurally identical, to a target cannabinoid interacts with cannabinoid receptor using routine screening assays, such as the ligand binding assays described by Devane et al. (1988) Mol Pharmacol 34:605-613. Various cannabinoid analogs are known in the art and some exemplary beverage compositions of the present invention may comprise such cannabinoid analogs. For example, PCT Publication WO2017/132526 and U.S. Pat. No. 6,630,507, which are each hereby incorporated by reference in their entireties, describe various analogs of cannabidiol.

In some embodiments, the anti-inflammatory agent (e.g., cannabinoid) may be present in a single serving of the beverage composition in an amount of from about 1 mg to about 20 mg, or about 5 mg to about 20 mg, or about 1 mg to about 10 mg, or about 2 mg to about 10 mg, or about 5 mg to about 15 mg, or about 5 mg to about 10 mg. For example, the anti-inflammatory agent may be present in a single serving of the beverage composition in an amount of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg.

In various embodiments, the compounds described above may be in a pharmaceutically acceptable salt form which, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, magnesium, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and —N+(C₁₋₄alkyl)⁴ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some embodiments, the beverage may include an emulsifier. Cannabinoids are highly lipophilic, making these compounds difficult to dissolve in the aqueous liquids used to create beverages and poorly absorbed systemically. In some embodiments, the beverage may include an emulsifier to aid in the dissolution of cannabinoids. The emulsifier used in the beverages are not limited. For example, the emulsifier of various embodiments may be selected poloxamer 188, polysorbate 80, polysorbate 20, vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, polysorbate 85, polyglyceryl-6-dioleate, sorbitan monooleate, capmul MCM, maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate, lecithin and the like and combinations thereof. In some embodiments, beverages containing an emulsifier may further include a co-solvent such as, for example, co-solvents selected from the group consisting of ethanol, glycerol, propylene glycol, 1,3-propanediol, butylene glycol, erythritol, xylitol, mannitol, sorbitol, isomalt, polyethylene glycols, and the like and combinations thereof that further aid in the dissolution of the cannabinoid. The amount of emulsifier incorporated into may be determined by the person of ordinary skill in the art based on the type or emulsifier, the presence or absence of a cosolvent, and other considerations known in the art. In various embodiments, the amount of emulsifier may vary from about 2 w/v % to about 30 w/v % or any range or individual concentration within this example range.

In some embodiments, the beverage may include a bioenhancer. As used herein, the term “bioenhancer” refers to an agent capable of enhancing bioavailability and efficacy of a drug with which it is co-administered, without any pharmacological activity of its own at the dose used. In some embodiments, the bioenhancers may also aid in the dissolution of the cannabinoid. Bioenhancers include, for example, P-glycoprotein inhibitors, compounds that reverse P-glycoprotein-mediated efflux, limit metabolism of active agents, increase gastric emptying time and intestinal motility, reduce degradation of the active agent by hydrochloric acid, modify cell membrane permeability, produce a cholagogue effect, modify the bioenergetics and thermogenic properties of the active agent, suppress first pass metabolism, and inhibit metabolizing enzymes, stimulate gamma glutamyl transpeptidase, enhance the uptake of amino acids, and the like and combinations thereof. In some embodiments, the bioenhancers may be herbal or neutraceutical bioenhancers. Examples of bioenhancers encompassed by the invention include piperine, quercetin, genistein, naringin, sinomenine, glycyrrhizin, nitrile glycosides cuminum cyminum, zingiver officinale, lysergol, allium sativum, aloe vera, and the like and combinations thereof.

In some embodiments, the bioenhancers may be liposomes, microspheres, nanoparticles, transferosomes, ethosomes, nanoemulsions, microemulsions, lipid based systems, polymeric micelle formulations, ketoprofen-loaded solid lipid nanoparticles, and the like, which may be made from beeswax, carnauba wax, or other natural waxes and solid lipids, and combinations thereof. In some embodiments, the bioenhancers may be liposomal enhancers such as, for example, Ginkgo biloba lipid-based systems, silybin lipid-based systems, ginseng lipid-based systems, hawthorn lipid-based systems, quercetin lipid-based systems, curcumin lipid-based systems, and the like and combinations thereof. In further embodiments, the bioenhancers may be capsaicin transferosomes, colchicine tranferosomes, vincristine tranferosomes, and the like and combinations thereof, which may find particular use as natural skin penetration agents.

In some embodiments, the bioenhancer may be a cyclic amine such as, for example, benzylamines, pyrrolidines, piperidines, morpholines, piperazines, and the like and derivatives thereof. In certain embodiments, the cyclic amine may be piperidine or a piperidine derivative such as N-methylpiperidine, N-ethylpiperidine, N-phenylpiperidine, pelletierine, sedamine, sedridine, allosedridine, dumetorine, solenopsins, isosolenopsins, andrachcinidine, cassine, prosopinine, prosophylline, morusimic acid, and the like and combinations thereof. In some embodiments, the cyclic amine may be isolated from or contained in an extract from a natural source such as pepper, sanguinarine, coptisine, goldenseal, and the like and combinations thereof. For example, piperine enhances bioavailability by modulating DNA receptor binding and cell signal transduction, while inhibiting efflux pumps that remove the active agent from cells. This inhibits drug metabolizing enzymes and stimulates absorption by stimulating gut amino acid transporters and inhibiting cellular pumps responsible for drug elimination from cells and intestinal production of glucuronic acid. Piperine also increases the absorption of the active agent in the gastrointestinal tract and inhibits enzymes responsible drug metabolism especially in the liver during first pass metabolism such as hepatic arylhydrocarbon hydrolase and UDP-glucuronyltransferase activities. Piperine modifies the rate of glucuronidation by lowering the endogenous UDP-glucuronic acid content and also by inhibiting transferase activity. Piperine inhibits P-glycoprotein and cutochrome P450 3A4, also CYPIA1, CYPIB1, CYP1B2, CYP2E1, CYP3A4, among others and makes target receptors more responsive to drugs, acting as receptors for drug molecules, increasing GIT vasculature by vasodilation to increase the absorption of drugs, modulation of cell membrane dynamics which increases transport of drugs across the cell membranes.

The amount of bioenhancer in a single serving of the beverage compositions of the present invention may be from about 0.05% to about 20% (w/w), relative to the total amount of the composition or in some embodiments, from about 0.1% to about 10% (w/w), relative to the total weight of the composition, from about 0.1% to about 5% (w/w), relative to the total amount of the composition, from about 0.1% to about 2% (w/w), relative to the total amount of the composition, or any range or individual concentration encompassed by these example ranges.

In some embodiments, the beverages may include additional ingredients such as vitamins (e.g., vitamin A, vitamin C), electrolytes, protein, fiber, antioxidants, sweeteners, and functional ingredients, such as, calcium, sodium, potassium, iron, bicarbonate, caffeine, fiber, protein, taurine, ribose, omega 3, and the like and combinations thereof. Additional ingredients may further include one or more grains such as, for example, barley, wheat, rye, buckwheat, bare wheat, rice, soybeans, black soybeans, broad beans, kidney beans, red beans, cowpeas, peanuts, peas, mung bean, buckwheat, corn, white sesame, black sesame, persimmon, kinuwa, and the like and combinations thereof. In certain embodiments, the beverages may include additional flavorings from fresh ingredients or extracts. Non-limiting examples of flavorings branded beverage concentrate, plum, blueberry, mango, cherry, grape, kiwi, strawberry, lemon, lime, passion fruit, apple, melon, tangerine, raspberry, orange, pomegranate, pineapple, coconut, grapefruit, acai, watermelon, peach, or any combination thereof.

In some embodiments, the composition may include an antioxidant. Such antioxidant may be, for example, butylated hydroxytoluene, ascorbic acid, ascorbyl palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant may be present in a concentration of about 0.01% (w/w) to about 1% (w/w) of the total composition or any individual concentration encompassed by this example range.

For example in some embodiments, the beverages may include a buffering agent. Buffering agents may be used to provide drug stability, to control the therapeutic activity of the drug substance, and to prevent the initial discomfort associated with injections. Suitable buffers include, but are not limited to, sodium bicarbonate, sodium citrate, citric acid, sodium phosphate, and the like and combinations thereof. When one or more buffers are utilized in the formulations of the invention, they may be combined with a pharmaceutically acceptable vehicle and present in an amount from about 0.1% (w/w) to about 20% (w/w).

In certain embodiments, the beverages may be sparkling. As used herein, “sparkling” means that the beverage contains dissolved or infused gas bubbles of carbon dioxide, nitrogen, or a mixed gas.

The beverage compositions of the present invention may be consumed at any suitable temperature, including warm (e.g., at 30-70° C.), ambient temperature (e.g., at 20-30° C.), or chilled (e.g., at 4-19° C.). Advantageously, consumption of chilled beverages may enhance sleep by encouraging the body to start lowering its core temperature.

Broadly speaking, the beverage compositions of the present invention may be prepared by combining together the components of the beverage composition as described herein with an aqueous liquid (e.g., water, alcohol, fruit juice, or a combination thereof), at a temperature and for a time sufficient to provide a consumable beverage. For example, in some embodiments, the components of the beverages may be dissolved, suspended, dispersed or otherwise mixed in a selected carrier or vehicle, such as water, a hydroalcoholic mixture or fruit juice.

Beverage compositions of the present invention may be provided in any suitable form. For example, in one embodiment, the beverage composition is provided as a liquid solution or emulsion comprising an aqueous (e.g., water or a hydroalcoholic mixture) hibiscus extract as described elsewhere herein (e.g., an extract of Hibiscus sabdariffa, Hibiscus acetosella, or other species of hibiscus, or a combination thereof), optionally comprising one or more additional components as described herein (e.g., a extract from another plant, a cannabinoid, bioenhancer, or the like, or combinations thereof) to provide a ready-to-drink beverage composition. Ready-to-drink beverage compositions of the present invention may be packaged in a suitable container, such as a plastic or glass bottle, can, box (e.g., Tetra Pak®), bag in box, jar, or the like. The container may be any suitable volume, e.g., 200 mL, 300 mL, 400 mL, 500 mL, 600 mL, 700 mL, 800 mL, 900 mL, 1 L, 1.25 L, 1.5 L, 2 L, or more. The container may comprise a single serving of the beverage composition, or it may comprise more than one serving (e.g., 2, 3, 4, 5 or more servings) of the beverage composition or less than one serve of the beverage (e.g., ¼, ⅓, ½, etc. servings) composition. A single serving of the beverage composition may be 100 mL, 200 mL, 300 mL, 400 mL, 500 mL or more.

In other embodiments, the beverage composition may be provided as a dry composition, which may be combined with an aqueous liquid (e.g., 200 mL, 300 mL, 400 mL, 500 mL, 600 mL, 700 mL, 800 mL, 900 mL, 1 L, 1.25 L, 1.5 L, 2 L, or more) to provide a ready-to-drink beverage. Dry compositions may be prepared, for example, by providing a composition comprising dried hibiscus leaves and/or flowers together with magnesium, which generate a hibiscus extract when treated with an aqueous liquid. Alternatively, dry compositions may comprise a dried (e.g., dehydrated) hibiscus extract (e.g., an extract of a hibiscus leaf, flower and/or other part of the plant), which has been combined with magnesium and may be reconstituted in an aqueous liquid or emulsion to produce a beverage of the present invention. In an embodiment, the beverage composition is provided as a dry composition comprising a hibiscus extract as described elsewhere herein (e.g., an extract of Hibiscus sabdariffa, Hibiscus acetosella, or other species of hibiscus, or a combination thereof), optionally comprising one or more additional components as described herein (e.g., a extract from another plant, a cannabinoid, bioenhancer, or the like, or combinations thereof). Such dry composition may be mixed with an aqueous liquid (e.g., water or a hydroalcoholic mixture) to provide a ready-to-drink beverage of the present invention. In some embodiments, the dry composition may be in the form of a powder (e.g., a loose powder or compressed powder), or capsule (e.g., an encapsulated powder) that, when combined with an aqueous liquid produces a liquid beverage composition as disclosed herein. For example, a loose or compressed powder may be dispersed and/or dissolved in an aqueous liquid (e.g., water or a hydroalcoholic liquid) to provide an infused liquid, emulsion or solution for consumption. In some embodiments, dispersion or dissolution of a loose or compressed powder may result in spontancous effervescence. In some embodiments, the dry compositions of the present invention may be ingested directly (i.e., without dissolution or dispersion in a liquid). Such dry beverage compositions may be provided in any suitable container, including but not limited to, a sachet, tea bag, tube, box, canister, or the like.

The present invention also provides kits for preparing beverage compositions as disclosed here. The kit may include, for example, the plant leaf and/or flower (e.g., in dried form), or an extract thereof, and the mineral, each packaged or formulated individually, or packaged or formulated in combination. Thus, the plant leaf and/or flower (e.g., hibiscus leaf and/or flower), or extract thereof may be present in first container, and the mineral (e.g., magnesium) may be present in a second container. The container or containers may be placed within a package, and the package may optionally include instructions for use. In another embodiment, the plant leaf and/or flower (e.g., hibiscus leaf and/or flower), or extract thereof, and the mineral (e.g., magnesium) may be provided in dry from in a container (e.g., a sachet), which may be provided with instructions for combining with an aqueous liquid to prepare a beverage composition of the present invention. The kits may optionally comprise instructions describing a method of using the beverage composition in one or more of the methods described herein (e.g., for reducing blood pressure).

In addition to the beneficial effects of the hibiscus extracts and magnesium contained within the beverages of the present invention, substitution of sugar-sweetened or artificially-sweetened beverages with the plant-based beverages of the present invention may offer significant cardiovascular health benefits, including beneficial effects on blood pressure. Consumption of sugar-sweetened beverages and artificially-sweetened beverages are associated with risk of hypertension and other cardiometabolic diseases, stroke, and mortality in adults. Dose-response analyses have shown linearly increased risk of hypertension with the increasing consumption of sugar-sweetened or artificially-sweetened beverages, in addition to an association of increasing levels of sugar-sweetened or artificially-sweetened beverage consumption per serving per day with significantly higher risk levels of hypertension. In addition to the adult population, consumption of sweetened beverages is associated with an increased risk of hypertension among children and adolescents. In non-hypertensive and undiagnosed hypertensive individuals, effective dietary interventions can yield improvements in blood pressure and prevent progression to clinical hypertension. In stage I hypertension, dietary changes can serve as an early intervention option prior to initiating drug therapy.

Thus, the beverage composition of the present invention may provide a useful alternative to energy drinks and other beverages that have a high content of sugar and/or high-fructose corn syrup and/or artificial sweeteners (e.g., acesulfame K, aspartame, advantame, cyclamates, neotame, saccharin, sucralose, and the like). The beverage composition of the present invention may also provide a useful alternative to beverages that contain high amounts of caffeine and/or preservatives (e.g., sodium benzoate, potassium benzoate, potassium sorbate), which can also elicit adverse effects. For example, the high caffeine content of some energy drinks can cause jitteriness, increase heart rate, gastrointestinal comfort, insomnia, anxiety, heart complications and/or dehydration in some consumers. Thus, in some embodiments, the beverage compositions of the present invention are substantially free of added caffeine or other stimulants. In some embodiments, the beverage compositions of the present invention are substantially free of added sugar and/or high-fructose corn syrup. In some embodiments, the beverage compositions of the present invention are substantially free of artificial sweeteners. In some embodiments, the beverage compositions of the present invention are substantially free of added preservatives.

The beverage compositions of the present invention may be useful in modulating blood pressure in a subject, e.g., by lowering or preventing an increase in blood pressure. Thus, the present invention provides a method of reducing blood pressure or preventing an increase in blood pressure in a subject, comprising the subject consuming a beverage composition containing hibiscus and an effective amount of magnesium. As used herein, an “effective amount” refers to an amount of magnesium sufficient to reduce blood pressure in the subject, preferably a safe reduction (e.g., a healthy, gradual reduction) of the blood pressure of the subject. The subject may be normotensive (i.e., SBP<120 mm Hg and DBP<80 mm Hg), or have prehypertension (i.e., SBP 120-129 mm Hg and DBP>80 mm Hg, or have frank hypertension (i.e., SBP≥130 mm Hg or higher and DBP≥80 mm Hg). In an embodiment, the present invention provides a method of reducing a high blood pressure, comprising the subject consuming a beverage composition containing a hibiscus extract and an effective amount of magnesium, as disclosed herein. As used herein, subjects with “high” blood pressure encompasses prehypertensive and hypertensive subjects, i.e., subjects having SBP≥120 mm Hg and DBP≥80 mm Hg. Such reduction may result in a reduction to the normotensive range (i.e., SBP<120 mm Hg and DBP<80 mm Hg) or otherwise. Thus, the beverage compositions of the present invention may modulate blood pressure so as to support a healthy (or normal) blood pressure in a subject. A “healthy” or “normal” blood pressure may vary depending what is safe and healthy for the individual and may include, but is not limited to, a normotensive blood pressure (i.e., SBP<120 mm Hg and DBP<80 mm Hg).

In some embodiments, the beverage compositions of the present invention promote healthy longevity in a subject. For example, high blood pressure, which is associated, for example, with cardiovascular disease and metabolic syndrome, may lead to premature death. Thus, the term “healthy longevity” (or “healthy ageing”) refers to the subject living out their natural life span.

A single serving of the beverage composition may be consumed at a suitable frequency to deliver an effective amount of magnesium to the subject. For example, the subject may consume a single serving of the beverage composition once daily or in multiple (e.g., two three, four, etc.) daily doses, or at a greater or lesser frequency as deemed necessary to achieve one or more benefits of the invention, e.g., to safely reduce or maintain blood pressure to a preferred range (e.g., SBP<120 mm Hg and DBP<80 mm Hg) in accordance with accepted medical guidance. Subjects who are receiving medical treatment for hypertension may benefit from consulting with a health care professional in order to achieve safe and effective, gradual and healthy reductions in their blood pressure. The frequency of consumption of the beverage may be daily, or several (two, three, four, or more) times daily, based upon individual health needs and tolerance, for a duration sufficient to reduce or prevent an increase in (and, optionally, maintain) blood pressure. The beverage may be consumed for a discrete period of time (e.g., a week, a month, a year) or on an ongoing basis.

In addition to lowering blood pressure, the beverage compositions disclosed herein may provide other health benefits associated with hibiscus and/or magnesium, including but not limited to, preventing or reducing the risk of: cardiovascular disease (CVD), cancer (e.g., rectal adenomas), obesity, cardiometabolic disease, kidney stones, neuromuscular, cardiac or nervous disorders, stroke, coronary heart disease, heart failure, hyperlipidemia, hyperglycemia, blood fat, blood sugar, chronic kidney disease, atherosclerosis, endothelial dysfunction, and arterial stiffening, erectile dysfunction, premenstrual syndrome, preeclampsia, preterm birth, cerebral palsy, osteoporosis, asthma, chronic obstructive pulmonary disease (COPD), cystic fibrosis, coronary artery disease, myocardial infarction, arrhythmia, heart failure, vascular calcification, atopic dermatitis, psoriasis, cataract, glaucoma, hearing loss, liver cirrhosis, non-alcoholic fatty liber, hepatic cancer, insulin resistance, prediabetes and type 2 diabetes mellitus. Other beneficial effects may include reduced stress and/or anxiety, reduced muscle tension and/or cramps, reduced incidence of headaches and/or migraines, improved/elevated mood, improved sleep quality and/or reduced insomnia, improved cognitive function and/or academic performance, improved exercise performance and/or sexual performance, reduced dreaminess, sleep induction, calming, mood elevating, appetite-reducing effects, weight loss effects, and/or immune support.

In some embodiments, the beverage compositions disclosed herein may mitigate (e.g., slowing, halting or preventing) the progression of sub-clinical cardiovascular, metabolic and/or cardiovascular disorders.

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

A beverage containing organic hibiscus tea, Mg-ascorbate, and licorice root extract as a sweetener and a bioenhancer can be produced. This beverage includes at least as much vitamin C as orange juice with 0 sugar, compared to beverages like orange juice that contain about 20 g of sugar in orange juice, and is high in iron. In addition, the beverage may lower blood pressure. Thus, the beverage may have a positive impact on women's health as well as patients exhibiting the symptoms of, or having been diagnosed with hypertension and other metabolic disorders.

### Example 2

A liquid (tea) and a dry powder beverage composition of the invention may be prepared in accordance with Tables 2 and 3 below, respectively.

TABLE 2
Liquid hibiscus-magnesium tea formulation (4 × 250 mL servings)

| Component | Amount | |
|---|---|---|
| Hibiscus Extract | 3.0-25 | g |
| Glycyrrhizin | 0.001-25 | g |
| Magnesium | 320-1,600 | ng |
| Vitamin C | 525-2,500 | mg |
| Quercetin | 1.0-5.0 | g |
| Curcumin | 1.6-6.5 | g |
| Piperine | 1.6-88 | mg |
| Iron | 32-88 | mg |
| Water | 950-995 | g |

TABLE 3
Dry powder hibiscus-magnesium formulation (4 × 16 g servings)

| Component | Amount | |
|---|---|---|
| Hibiscus Extract | 3.0-25 | g |
| Glycyrrhizin | 0.001-25 | g |
| Magnesium | 320-1,600 | mg |
| Vitamin C | 525-2,500 | mg |
| Quercetin | 1.0-5.0 | g |
| Curcumin | 1.6-6.5 | g |
| Piperine | 1.6-88 | mg |
| Iron | 32-88 | mg |

The beverage compositions of Tables 2 and 3 may be consumed in an amount sufficient to achieve the RDA of magnesium (e.g., one serving per day). These beverage compositions may include at least as much vitamin C and iron as an equivalent amount of orange juice, while being free of added sugar. In addition, the beverage may lower blood pressure, and provide other health benefits, such as mitigating progression of sub-clinical metabolic and/or cardiovascular disorders, and/or developing an enhanced sense of well-being. Thus, the beverage may have a positive impact on adolescent's, men's and women's physical, sexual and mental health, including support of cognitive function and healthy aging, as well as supporting patients diagnosed with hypertension and/or other cardiometabolic disorders.

1. A beverage composition comprising a hibiscus extract and magnesium.

2. The beverage composition of claim 1, wherein the hibiscus extract is a Hibiscus sabdariffa or Hibiscus acetosella flower extract.

3. The beverage composition of claim 1, wherein the composition comprises magnesium in an amount of from about 30 mg to about 420 mg.

4. The beverage composition of claim 1, wherein the magnesium is in the form of magnesium carbonate, magnesium chloride, magnesium hydroxide, magnesium oxide, magnesium phosphate, magnesium stearate, magnesium sulfate, magnesium ascorbate, magnesium citrate, magnesium taurate, or a combination thereof.

5. The beverage composition of claim 1, further comprising less than 140 mg sodium.

6. The beverage composition of claim 1, further comprising an anti-inflammatory agent.

7. The beverage composition of claim 6, wherein the anti-inflammatory agent is a cannabinoid.

8. The beverage composition of claim 7, wherein the cannabinoid is an amount of from about 1 mg to about 20 mg.

9. The beverage composition of claim 6, further comprising a bioenhancer.

10. The beverage composition of claim 9, wherein the bioenhancer is in an amount of about 1 mg to about 20 mg.

11. The beverage composition of claim 1, wherein the composition is substantially free of caffeine.

12. The beverage composition of claim 1, wherein the composition is substantially free of sugar and/or high fructose corn syrup.

13. The beverage composition of claim 1, wherein the composition is substantially free of an artificial sweetener.

14. The beverage composition of claim 1, wherein the composition is substantially free of a preservative.

15. The beverage composition of claim 1, wherein the composition is a liquid ready-to-drink composition.

16. The beverage composition of claim 1, wherein the composition is a dry composition.

17. A process for preparing a beverage composition of claim 1, comprising combining a hibiscus extract and magnesium.

18. The process of claim 17, further comprising combining the hibiscus extract and magnesium with an aqueous liquid or emulsion.

19. A use of the beverage composition of claim 1, comprising reducing blood pressure or preventing an increase in blood pressure in a subject.

20. A use of the beverage composition of claim 1, comprising preventing or reducing the risk of developing a disease or condition selected from the group consisting of cardiovascular disease (CVD), cancer (e.g., rectal adenomas), obesity, cardiometabolic disease, kidney stones, neuromuscular, cardiac or nervous disorders, stroke, coronary heart disease, heart failure, hyperlipidemia, hyperglycemia, blood fat, blood sugar, chronic kidney disease, atherosclerosis, endothelial dysfunction, and arterial stiffening, erectile dysfunction, premenstrual syndrome, preeclampsia, preterm birth, cerebral palsy, osteoporosis, asthma, chronic obstructive pulmonary disease (COPD), cystic fibrosis, coronary artery disease, myocardial infarction, arrhythmia, heart failure, vascular calcification, atopic dermatitis, psoriasis, cataract, glaucoma, hearing loss, liver cirrhosis, non-alcoholic fatty liber, hepatic cancer, insulin resistance, prediabetes and type 2 diabetes mellitus, or mitigating the progression of sub-clinical cardiovascular, metabolic and/or cardiovascular disorders, in a subject.

21. A use of the beverage composition of claim 1, comprising reducing stress and/or anxiety, reducing muscle tension and/or cramps, reducing incidence of headaches and/or migraines, improving/elevating mood, improving sleep quality and/or reducing insomnia, improving cognitive function and/or academic performance, improving exercise performance and/or sexual performance, reducing dreaminess, inducing sleep, calming, elevating mood, reducing appetite, weight loss, and/or providing immune support, in a subject.

22. A method of reducing blood pressure or preventing an increase in blood pressure in a subject, comprising the subject consuming a beverage composition containing a hibiscus extract and an effective amount of magnesium.

23. The method of claim 19, wherein the subject has hypertension or prehypertension.

24. A method of preventing or reducing the risk of developing a disease or condition selected from the group consisting of cardiovascular disease (CVD), cancer (e.g., rectal adenomas), obesity, cardiometabolic disease, kidney stones, neuromuscular, cardiac or nervous disorders, stroke, coronary heart disease, heart failure, hyperlipidemia, hyperglycemia, blood fat, blood sugar, chronic kidney disease, atherosclerosis, endothelial dysfunction, and arterial stiffening, erectile dysfunction, premenstrual syndrome, preeclampsia, preterm birth, cerebral palsy, osteoporosis, asthma, chronic obstructive pulmonary disease (COPD), cystic fibrosis, coronary artery disease, myocardial infarction, arrhythmia, heart failure, vascular calcification, atopic dermatitis, psoriasis, cataract, glaucoma, hearing loss, liver cirrhosis, non-alcoholic fatty liber, hepatic cancer, insulin resistance, prediabetes and type 2 diabetes mellitus, or mitigating the progression of sub-clinical cardiovascular, metabolic and/or cardiovascular disorders, in a subject, the method comprising the subject consuming a beverage composition containing a hibiscus extract and an effective amount of magnesium.

25. A method of reducing stress and/or anxiety, reducing muscle tension and/or cramps, reducing incidence of headaches and/or migraines, improving/elevating mood, improving sleep quality and/or reducing insomnia, improving cognitive function and/or academic performance, improving exercise performance and/or sexual performance, reducing dreaminess, inducing sleep, calming, elevating mood, reducing appetite, weight loss, and/or providing immune support, in a subject, the method comprising the subject consuming a beverage composition containing a hibiscus extract and an effective amount of magnesium.
